(19) United States
(12) Patent Application Publication
(10) Pub. No.: US 2011/0218241 A1
(43) Pub. Date: Sep. 8, 2011

(54) ANTIVIRAL EPICATECHINS, EPICATECHIN OLIGOMERS, OR THIOLATED EPICATECHINS FROM THEOBROMA CACAO FOR TREATMENT OF GENITAL WARTS

(75) Inventors: Daniel PRESTON, Brooklyn, NY (US); Randall B. MURPHY, Glenmoore, PA (US)

(73) Assignee: CACAO BIO-TECHNOLOGIES, LLC, Brooklyn, NY (US)

(21) Appl. No.: 13/039,760

(22) Filed: Mar. 3, 2011

Related U.S. Application Data
(60) Provisional application No. 61/311,317, filed on Mar. 6, 2010.

(51) Int. Cl. A61K 31/355; A61K 31/353; A61P 31/12; A61B 18/12; A61B 18/04; A61B 18/20; A61B 18/02

(52) U.S. Cl. 514/458; 514/456; 606/41; 606/28; 606/2; 606/20

(57) ABSTRACT

Epicatechins, Epicatechin Oligomers, or Thiolated Epicatechins are applied (A) directly to a genital wart in the form of a cream, ointment, paste or solution, (B) directly to the genital wart wherein such cream, ointment, paste or solution contains as an additional active ingredient a skin permeabilizing agent, (C) following electrosurgical resection or removal of the genital wart in such form of a cream, ointment, paste or solution, (D) following chemical resection or extirpation of the genital wart in such form, (E) following surgical resection or removal of the genital wart in such form, wherein said Epicatechins, Epicatechin Oligomers, or Thiolated Epicatechins both provide antiviral activity against multiple strains of human papilloma virus (HPV) and promote healing following resection polymers contained in a vehicle. Disclosed are the compositions, therapeutical kits containing such composition, methods of treatment using such composition, and methods of enhancing the stability of such composition.

### CROSS REFERENCE TO RELATED APPLICATION

This application claims the priority of U.S. provisional patent application Ser. No. 61/311,317, filed Mar. 6, 2010, and hereby incorporates the entire contents of said provisional application into this disclosure by reference.

### SEQUENCE LISTING

The instant application contains a Sequence Listing which has been submitted in ASCII format via EFS-Web and is hereby incorporated by reference in its entirety. Said ASCII copy, created on May 12, 2011, is named Y4132015.txt and is 1,731 bytes in size.

### FIELD OF THE INVENTION

This invention relates to the topical application of compositions containing epicatechin oligomers and catechins and/or catechins derivatives to the human or animal dermis or mucosa for the amelioration of infection by the Human Papilloma Virus (HPV). The invention further relates to a method of treatment following excision of genital ulcers and warts caused by HPV, wherein application of said compositions to the affected area, is virucidal and improves healing of the area. It further relates to such a method of treatment when said genital warts are located within the human vagina or rectum. The invention provides preparations and kits for this purpose.

### BACKGROUND OF THE INVENTION

### General

Cervical cancer is the second most common type cancer in women worldwide. Incidence rates vary from about 10 per 100 000 per year in many industrialized nations to more than 40 per 100,000 in some developing countries. 75% of new cases are currently diagnosed in the developing parts of the world. Epidemiologic studies have shown that the association of Human Papillomavirus (HPV) with cervical neoplasia is strong and independent of other risk factors

More than 35 distinct HPV types are known to infect the genital tract, complicating its detection. Twenty or more of these HPV types are cancer-associated. HPVs appear to represent the most common sexually transmitted agent studied to date. Studies of cytologically normal women suggest that 20%-40% of sexually active young women have detectable HPV infection and that prevalence decreases with age. In most studies, HPV 16 has been found to be the most prevalent HPV type. The prevalence of HPV in the general population has been estimated to be between 13% and 20% in Thailand, the Philippines, Paraguay, Brazil, and Colombia. Prevalence in western Europe and the United States is generally less than 10% at 40 years of age or older.

It is estimated from that more than 99% of all cervical cancers worldwide are positive for so-called high-risk HPV strains.

The vaginal cavity, including the vagina and cervix, provides a unique site for viral infection as well as drug delivery. There are multiple anatomical structures which comprise the internal and external female genital tract including the clitoris, labia minora and corpus spongiosum (vestibular) erectile tissue, vagina, peri-urethral glans, urethra, Halban's fascia, anterior formix erogenous zone, pubococcygeus muscle and cervix.

The vagina consists of a tube of autonomically-innervated smooth muscle (longitudinal outer, inner circular layer) lined by stratified squamous epithelium and a sub-dermal layer rich in capillaries. The vaginal wall consists of an inner glandular mucous type stratified squamous cell epithelium supported by a thick lamina propia. This epithelium undergoes hormone-related cyclical changes including slight keratinization of the superficial cells during the menstrual cycle. Deep in the epithelium lies the smooth muscles of the muscularis. There is a deeper surrounding fibrous layer above the muscularis which provides structural support to the vagina and is rich is collagen and elastin to allow for expansion of the cavity. Three sets of skeletal muscles surround the vagina including the ischiocavernosum, bulbocavernosus, transverse perinei and levator ani and pubococcygeus muscles.

Women are vulnerable to diseases of the genital tract as the lining of the vagina is a permeable mucous membrane. Intercourse, lack of lubrication during intercourse, changes in the cervix during the menstrual cycle, and asymptomatic infections facilitate the transmission of infection to women. Prepubertal girls and adolescents are particularly vulnerable because their vaginal and cervical tissues may be less mature and are more readily penetrated by organisms (e.g., chlamydia and gonococcus). Postmenopausal women are more likely than younger women to get small abrasions in the vagina during sexual activity as a result of thinning of the tissue and dryness. Women who already have an infection (particularly one that causes genital lesions) are more likely to acquire or transmit another sexually transmitted disease (STD), including HIV.

An association of HPV infection with head and neck cancers is also clear. Certain cancers of the oral cavity, pharynx, and larynx are associated with HPV infection. Again, HPV-16 is the most prevalent type.

### The HPV Virus

Human papillomavirus (HPV) is a DNA virus which belongs to the family Papillomaviridae, with more than 100 types currently sequenced all with the potential to infect squamous epithelia Low risk mucosal human papillomaviruses such as HPV6 and HPV 11 cause genital warts, while high-risk HPVs such as HPV16 and HPV 18 cause intraepithelial lesions that can progress to invasive cell carcinoma.

Depending on the geographical region, 70% of human cervical cancers are associated with infections by high-risk HPV16 and HPV18 (7). In Brazil amongst women with HIV who had cytological abnormalities HPVs-16 and 81 were the most prevalent (14.1%) and were followed by HPVs 52, 35, 62, 33, 53, 56, 66, 70, 18, 58, 6b, 11, 31, 39, 40, 61, 71, 32, 54, 59, 67, 68, 85, and 102

HPV genomes contain six to eight open reading frames carried on one strand of DNA. All HPVs have a common genomic organization and encode 8 proteins: E1, E2, E4, E5, E6, and E7 (early) and L1 and L2 (late). Among the first viral genes expressed following infection are the replication proteins, E1 and E2, which have been shown to form a complex and bind to the viral origin sequences. E4 and E5 are believed to regulate late viral functions although their role is not clearly understood; E6 and E7 are oncoproteins; and L1 and L2 are structural proteins. The E6 and E7 oncoproteins of the high-risk strains are the main contributors to malignant transformation. Stable replication of the HPV-31 genome requires the expression of E6 and E7.

### The HPV E7 Protein

The HPV E7 proteins act by binding to members of the retinoblastoma (Rb) family of proteins, which allows cells to rapidly progress into S phase, a strategy common to other DNA viruses such as SV40 and adenovirus. Specifically, these E7 Rb targets are p105Rb, p107, and p130. The unphosphorylated form of the Rb protein binds to E2F/DP1 heterodimers and recruits histone deacetylase (HDAC) complexes to repress transcription from promoters containing E2F binding. As many as 11 different HDACs have been identified, and the most extensively studied are human HDAC1 and HDAC2. HDACs 1 and 2 usually associate with cellular DNA binding proteins that recruit them to genomic regions as well as modulate their deacetylase activities. HDACs repress transcription from promoters through deacetylation of lysine residues present in the N-terminal tails of core histone proteins. This deacetylation results in the unmasking of a positive charge on the lysine residue, resulting in a tight interaction between the histone proteins and the DNA. The tight binding then leads to heterochromatin formation and repression of transcription. In addition to this mechanism, the Rb family members can also interact with a number of other transcriptional activators, including c-jun and chromatin remodeling proteins.

### The HPV E6 Protein

The HPV E6 oncoproteins are small zinc-binding proteins with conserved overall structure but diverse activities, and considerable effort has been directed toward establishing their cellular targets. E6 is a small protein (150 amino acids) considering the number of interacting partners. In particular, it is known that E6 interacts with an LXXLL peptide sequence found on the cellular E3 ubiquitin ligase E6AP and together with E6AP binds to the p53 tumor suppressor protein, resulting in its ubiquitin-mediated degradation by the proteasome. E6 proteins have also been reported to target the degradation. A group of cellular proteins that interact with cancer-associated E6 proteins contain PDZ domains and bind the carboxy-terminal five amino acids of E6 that constitute a PDZ ligand consensus sequence. Cellular targets include DLG1 (human discs large homolog) and Scribble (that are tumor suppressors in Drosophila melanogaster), MUPP1, and membrane-associated guanylate kinase homologs with inactive kinase domains MAGI-1, MAGI-2, and MAGI-3. There are also three tyrosine phosphatases that contain PDZ domains that might be targeted by E6: PTPN3, PTPN4, and PTPN13. Association with E6 has been shown to result in instability of the PDZ-containing proteins in vitro.

Additionally, E6 is responsible for transcriptional activation of the telomerase reverse transcriptase (TERT) gene, which is the catalytic subunit of the enzyme telomerase. In HPV-infected cells, increased telomerase activity due to TERT transcription is believed to play a role in maintaining the telomeric repeats at chromosomal termini, allowing cells to avoid replicative senescence and become immortal.

Interestingly, the HPV E6 protein can both sensitize and protect cells from TNF, depending on the level of E6 expressed. The relationship between the effect of E6 on the cellular responses to TNF and to anti-Fas is complex, with low levels of E6 providing protection from TNF and high levels providing protection from Fas.

### The E1 Protein

The E1 protein, a helicase, has a normal cellular protein known as p80 as a target. This appears to be important in maintaining the virus in a stable system.

Interaction of HPV Virus with the Host

HPVs are persistent viruses that can remain in their hosts for long periods of time before causing any ill effects. Generally, the host reacts to viral pathogens by generating both humoral and cell-mediated responses. Humoral responses are typically antibody-mediated and involve the secretion of antibodies such as immunoglobulin A (IgA) and immunoglobulin G (IgG) by B lymphocytes. Cell-mediated responses, on the other hand, are carried out by immune effector cells such as dendritic cells (DCs), langerhans cells, natural killer (NK) cells, macrophages and T lymphocytes, which secrete a number of cytokines including interferons (INF) and tumor necrosis factor (TNF), and up-regulate the expression of Fas ligand (FasL) and TNF-related apoptosis-inducing ligand (TRAIL) on their cell surface.

### Current Treatment of HPV and Genital Warts

Most current treatments are ablative and directed to abnormal cells associated with HPV rather than the virus itself no direct antiviral treatment is available. Cryotherapy is used to freeze external warts by means of liquid nitrogen or dry ice applied directly on the lesions. Two cycles of freezing and thawing are usually performed. There is a variable response rate, and about one-fourth of the treated patients relapses. Cryotherapy requires special equipment, but is inexpensive and safe for the treatment of pregnant women. The use of a thermal cautery is also common.

The prevention of genital HPV infection is essential for reducing the prevalence of genital warts and abnormal Pap tests, as well as cervical cancer.

Recently, a highly effective vaccine was approved to prevent infections by four HPV types that together cause about 70% of cervical cancers (HPV-16 and HPV-18) and 90% of genital warts (HPV-6 and HPV-11) worldwide. However, women may remain exposed to the risk of becoming infected with some types of high-risk HPVs that can cause cervical cancer but are not targeted by the current vaccine.

Moreover, such vaccines are relatively expensive to produce and costly to administer. Thus, it may not be initially available to all women, especially those in developing countries. Many diseases that are controlled effectively in industrialized countries are controlled poorly at best in poor countries.

In this scenario, a topical microbicide, a compound that could block the full spectrum of genital HPV infections at the portal of entry, would be a useful complement to vaccination programs. However, just as has been the case with HIV it has proved to be extremely difficult to develop such a microbicide. In fact, as in HIV, it is known that heparin and other sulfated polysaccharides prevent the binding of HPV to the cell surface by mimicking cell surface sulfated glycoproteins. However, this approach does not seem to have been much utilized in the case of HPV.

Antiviral drugs are known which have activity against HPV, but generally they are little used because they have undesirable side effects and are thus reserved for more serious of life-threatening viral infections.

As an example the nucleoside phosphonates are useful. Of these compounds, the cytosine analog (S)-1-[3-hydroxy-2-(phosphonomethoxy)-propyl]cytosine (HPMPC) (cidofovir)

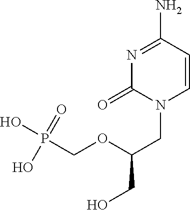

is highly effective in inhibiting HPV replication. It is effective in inhibiting herpes virus replication and is approved for treatment of cytomegalovirus (CMV) retinitis in AIDS patients. Cidofovir has broad-spectrum activity against virtually all DNA viruses, including herpes-, adeno-, polyoma-, papilloma- and poxviruses. However, it's substantial toxicity precludes it general use for HPV, although the toxicity associated with Cidofovir was markedly reduced when administered topically rather than intravenously.

Hostetler and his colleagues have introduced cidovir derivatives of reduced toxicity, which are lapidated and are slowly hydrolyzed in vivo, such as Octadecyloxyethyl-Phosphonomethoxyethylguanine (ODE-PMEG). These compounds have been principally developed to treat serious poxvirus infections of biomilitary significance (eg. Smallpox) but might have application in the HPV arena.

The antitumor nucleoside 5-fluorouracil is also effective against HPV but is highly toxic.

Other broad spectrum antiviral agents such as GSK983:

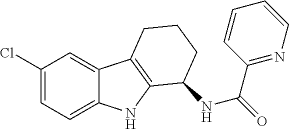

Have been shown to have anti HPV activity but have not been studied extensively in humans yet.

Certain cobalt complexes have also been reported to have activity against HPV in various model systems, as exemplified by CDC-96

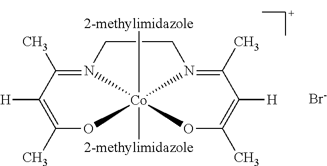

Indol-3-carbinol (I3C):

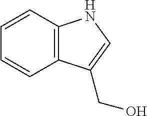

is a compound derived from cruciferous vegetables which possesses anti-HPV activity.

Various immunomodulators such as Imiquimod are also used to treated HPV lesions. This agent:

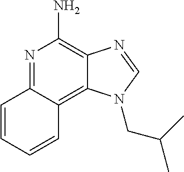

Upregulates the immune system by a variety of complex mechanisms which are not totally clear, presumably involving activation of TLR-7 receptors resulting in langerhans cells in the skin area becoming activated.

### Theobroma Cacao and Epicatechins

Chocolate, cocoa butter, and cocoa-flavoring ingredients are derived from the tropical fruit Theobroma cacao. Cocoa is ingested by many cultures and the discovery of its residue in ancient Mayan vessels suggests that humans have been consuming it, in some form, since at least 480 A.D. Common components of fresh cocoa beans (cotyledons) include theobromine, caffeine, flavinoid polyphenols, and saturated and monounsaturated fatty acids.

Flavonoids are a major class of plant polyphenolics, which comprises thousands of compounds such as flavonols, flavones, flavanones, flavanols, anthocyanins, dihydroflavonols, isoflavones and chalcones. Flavonoids are widely distributed in the plant kingdom, being present in a broad range of commonly consumed fruits and vegetables and plant-derived products such as cocoa, tea, and wine. Flavonols like quercetin mostly occur in foodstuffs as glycosides and, in general, the first step in their metabolism is likely to be deglycosylation before absorption in the small intestine; nonetheless they are generally well absorbed in man as well as in animals.

These beneficial actions of the flavinoids are due in part to their antioxidant activity. Antioxidant components are microconstituents present in the diet that can delay or inhibit lipid oxidation, by inhibiting the initiation or propagation of oxidizing chain reactions, and are also involved in scavenging free radicals. Food such as fruits, vegetables and grains are reported to contain a wide variety of antioxidant components, including phenolic compounds. These compounds are found to be well correlated with antioxidant potential

The interest in flavonoids has grown in the last fifteen years after the publication of several epidemiological studies showing an inverse correlation between dietary consumption of flavonoid-rich products and reduced incidence and mortality from cardiovascular disease and cancer Specifically epicatechins, such as epicatechin gallates originally identified in tea, have been reported to possess antimutagenic, antibacterial, antioxidant, antitumor and cancer preventive properties. Certain actions may also depend on pharmacological activities beyond their antioxidant properties. For example, tea polyphenols may induce apoptosis and are known to inhibit the growth of several cancer cell lines Polyphenols from other plant sources also inhibit the cellular expression of interleukin-8 and monocyte chemoattractant-1 when induced by the pro-inflammatory cytokine, tumor necrosis factor, and modulate of the pro-inflammatory cytokine interleukin-1. Epicatechin derivatives have also been shown to have antiviral and antibacterial activities.

Cacao products are rich in polyphenols such as epicatechin oligomers, as well as in other catechins and procyanidins. It has been reported that chocolate is a major source of catechins. 60% of the total phenolics in raw cocoa beans are flavanol monomers (epicatechin and catechin) and procyanidin oligomers (dimer to decamer). These compounds are well known in the prior art to combat free radicals, which are harmful to the human and animal body. Free radicals cause degenerative human diseases such as cancer, heart disease, and cerebrovascular disease through multiple mechanisms. In vitro studies demonstrated that the cacao flavinoid compounds have several biological activities, such as the ability to scavenge superoxide radicals and hydroxyl radicals, reduce lipid peroxyl radicals and inhibit lipid peroxidation Epicatechin oligomers in chocolate and cocoa are orally well absorbed and are metabolized and excreted as various conjugates. In a clinical study, cocoa powder supplementation was found to delay the oxidation of low density lipoprotein.

### Epicatechins

Epicatechins represent the basic monomeric unit of the proanthrocyanodins

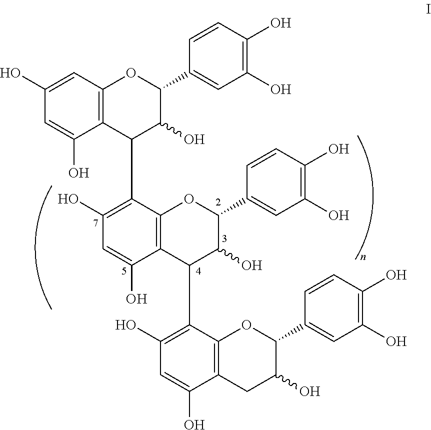

The basic molecular epicatechin unit is: typically it is present in the free from in cocao as the gallate ester derivative:

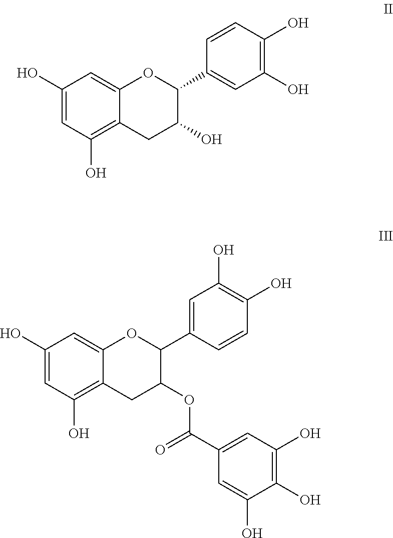

which has in itself been shown in prior art to be an extremely potent antioxidant material.

### SUMMARY OF THE INVENTION

It is an objective of this invention to provide a method and composition for the treatment of genital warts which are produced by infection with the HPV virus. Thus, according to a first feature, the invention relates to a composition for the treatment of genital warts comprising an extract of defined epicatechin oligomers

The present invention is a method of inhibiting papillomavirus infection.

An object of the present invention is to solve the technical problem concerned with healing after said genital warts are extirpated, either by use of low temperatures, in the form of cryosurgery, or by a high temperature cautery, typically of a monopolar or bipolar nature, or after laser surgery by which tissue is ablated by the use of a laser. Said laser will typically emit radiation in the infrared region of the electromagnetic spectrum, which locally produces heat and causes localized tissue destruction. Other wavelengths may also be employed.

According to a second feature, the invention relates to the use of said defined epicatechin oligomers as an active antiviral agent in compositions in the form of a kit which can be applied by the patient for intravaginal, intrarectal, or intracervical use. Said kit would consist of a syringe filled with the antiviral epicatechin composition which could be conveniently used to apply it to the required body cavity area. Refills for the syringe applicator would be provided within a packaged box or container which could conveniently be dispensed to the patient.

According to a third feature, the invention relates to a method of treatment wherein the mixture of defined antiviral epicatechin oligomers is contained in a base which is a hydrophylic gel, adjusted to the appropriate pH for intravaginal application. The base may also contain additional antibacterial, antifungal, or antiviral agents.

According to a fourth feature, the invention relates to a method of treatment wherein the mixture of defined antiviral epicatechin oligomers is contained in a base which is an oil-in water emulsion. This composition would be preferred for anorectal use upon genital warts. The base may also contain additional antibacterial, antifungal, or antiviral agents.

According to a fifth feature, the invention relates to a method of treatment wherein the mixture of defined epicatechin oligomers is contained in a base which is an oil-in water emulsion. This composition would be preferred for anorectal use upon genital warts. The base may also contain additional antibacterial, antifungal, or antiviral agents. The specific use for this preparation is for its ability to accelerate healing of the damaged tissue after extirpation rather than for its antiviral qualities per se.

According to a sixth feature, the invention relates to a method of treatment wherein the mixture of defined epicatechin oligomers is contained in a base which is a hydrophylic gel, adjusted to the appropriate pH for intravaginal application The base may also contain additional antibacterial, antifungal, or antiviral agents. The specific use for this preparation is for its ability to accelerate healing of the damaged tissue after extirpation rather than for its antiviral qualities per se.

These and other objectives are accomplished by the present invention, which provides methods and compositions for the treatment of genital warts by means of applying an effective amount of epicatechin oligomers and catechins and/or catechins derivatives preferably in a dermatologically acceptable carrier, to provide both antiviral and antioxidant activity which will promote healing.

Many embodiments incorporate other active ingredients with epicatechin oligomers and catechins and/or catechins derivatives. These include tocotrienols, ascorbate salts and esters, thiolic antioxidants such as glutathione, cysteine, ergothioneine, or ovothiol, resveratrol, ferulic acid, rosmarinic acid, caffeic acid, butylated hydroxyanisole, butylated hydroxytoluene, 2,6-diisopropylphenol, gallic acid, ethyl gallate, propyl gallate, isopropyl gallate, and benzyl gallate may be employed

In the preferred practice of the invention epicatechin oligomers and catechins and/or catechins derivatives is applied in admixture with an acceptable carrier or vehicle As noted supra, other ingredients, particularly as glutathione, cysteine, ergothioneine, lipoic acid, ovothiol, resveratrol, ferulic acid, rosmarinic acid, caffeic acid, butylated hydroxyanisole, butylated hydroxytoluene, 2,6-diisopropylphenol, gallic acid, ethyl gallate, propyl gallate, isopropyl gallate, and benzyl gallate are advantageously included in the compositions.

The amount of epicatechin oligomers and catechins and/or catechins derivatives necessary to bring about treatment is not fixed per se, and necessarily is dependent upon the identity and form of epicatechin oligomers and catechins and/or catechins derivatives employed, the amount and type of any additional ingredients used, particularly those that appear to exhibit synergistic effects, and the severity of the underlying viral infection and associate damage from a cautery or a cryosurgical or laser procedure. the user's skin type, and, where present, the severity and extent of the patient's skin damage.

In one embodiment, the composition contains from about 0.1% to about 5% by weight, preferably from more than 0.5% or 1.5% to about 3%, of epicatechin oligomers.

In another embodiment, the composition contains from about 0.1% to about 5% by weight, preferably from more than 0.5% or 1.5% to about 3%, catechins and/or catechins derivatives and epicatechin oligomers.

In yet another embodiment, the composition contains from about 0.1% to about 75% by weight, preferably from more than 1.5% or to about 30%, catechins and/or catechins derivatives and epicatechin oligomers.

In still another embodiment, the composition contains from about 0.1% to about 75% by weight, preferably from more than 1.5% or to about 40%, catechins and/or catechins derivatives and epicatechin oligomers.

In another embodiment, the composition contains from about 0.1% to about 75% by weight, preferably from more than 1.5% or to about 50%, catechins and/or catechins derivatives and epicatechin oligomers.

In yet another embodiment, the composition contains from about 0.1% to about 75% by weight, preferably from more than 1.5% or to about 30%, catechins and/or catechins derivatives and epicatechin oligomers in combination with an antioxidant selected from the group of ascorbic acid, including any of its pharmaceutically acceptable salts or pharmaceutically acceptable esters.

In still another embodiment, the composition contains from about 0.1% to about 75% by weight, preferably from more than 1.5% or to about 30%, catechins and/or catechins derivatives and epicatechin oligomers in combination with an antioxidant selected from the group of ergothioneine, lipoic acid, ovothiol, cysteine, penicillamine, N-acetylcysteine, cysteine C1-C30 alkyl ester, ebselen, sodium selenite, AD-4 thiol antioxidant, homocysteic acid, buthionine sulfoximine, selenocysteine, selenomethionine, bucillamine, N-acetylcysteine amide, 1,2-dithiol-3-thione, pyrrolidine dithiocarbamate, alkyl-2-thioacetate ester, alkyl 3-thiopropionate alkyl ester, alkyl-2-thiolpropionate alkyl ester, 3-(p-methoxyphenyl)-1,2-dithiol-3-thione; L-2-oxathiazalidine-4-carboxylate, alkyl-2-thiobutanoic ester, alkyl-4-thiobutanoic ester

In still an embodiment, the composition contains from about 0.1% to about 75% by weight, preferably from more than 1.5% or to about 30%, catechins and/or catechins derivatives and epicatechin oligomers in combination with an antioxidant selected from the group of resveratrol, caffeic acid, caffeoylgallic acid, ferulic acid, cyanidin, chrysin, delphinidin, bolodine, hesperetin, rutin, idealin, kaempferol, keracyanin, luteolin, malvidin, narengin, rosmarinic acid, pelargonidin peoniodin, porcyanidin C1, porcyanidin D1, porcyanidin D5, quercetin, quercetin-3-rutinoside, sinapic acid, taxifolin, or tetrapicatechin.

In still an embodiment, the composition contains from about 0.1% to about 75% by weight, preferably from more than 1.5% or to about 30%, catechins and/or catechins derivatives and epicatechin oligomers in combination with a Tocateienol.

### DETAILED DESCRIPTION OF THE INVENTION

This invention is based upon the unexpected finding that a composition of epicatechin oligomers and catechins and/or catechins derivatives is useful for treatment of tissue damage produced by a cautery, either of a thermal or laser nature, or a cryosurgical probe when these techniques are utilized to extirpate the genital wart.

### DEFINITIONS

As used herein, the term “epicatechin oligomers” encompasses the compounds of the structure:

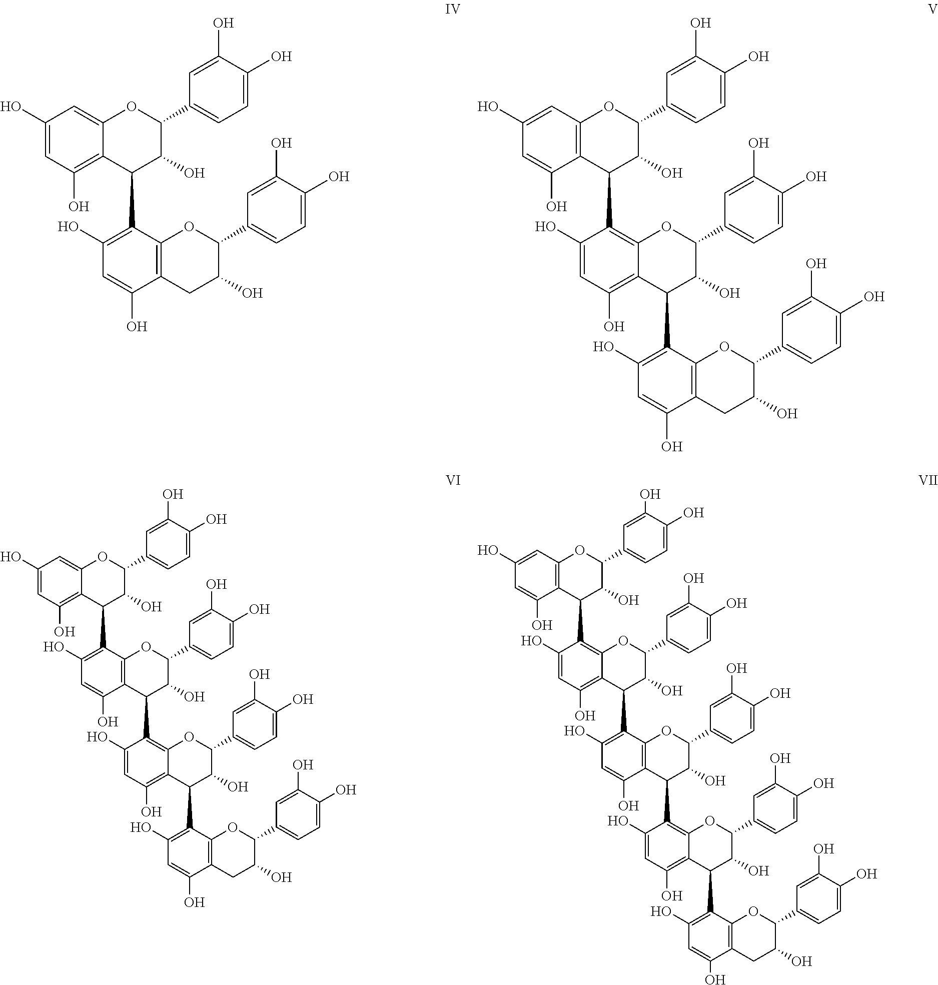

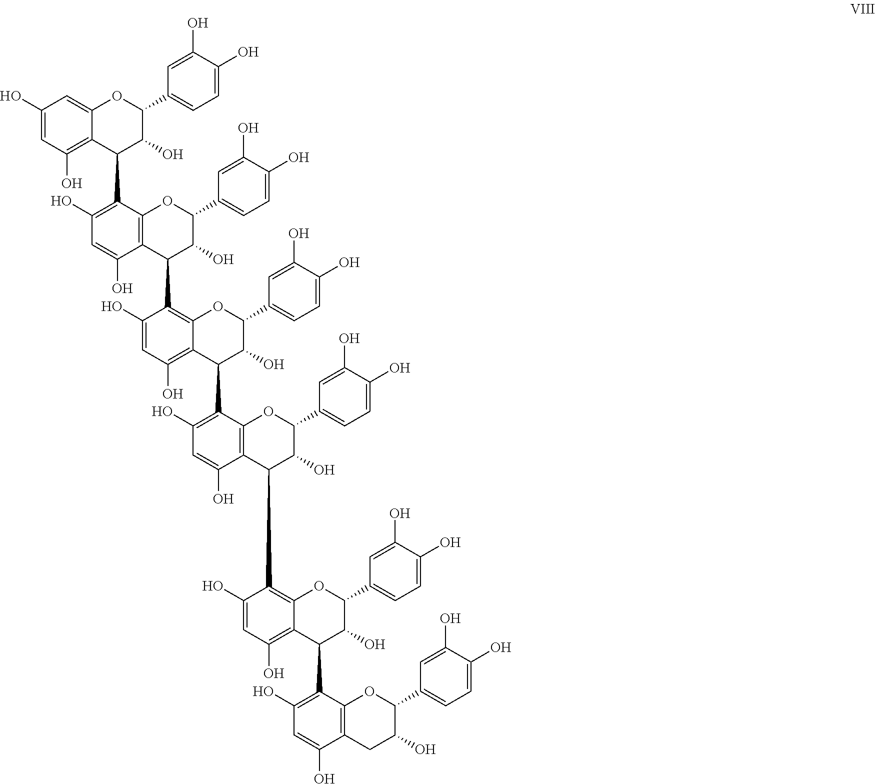

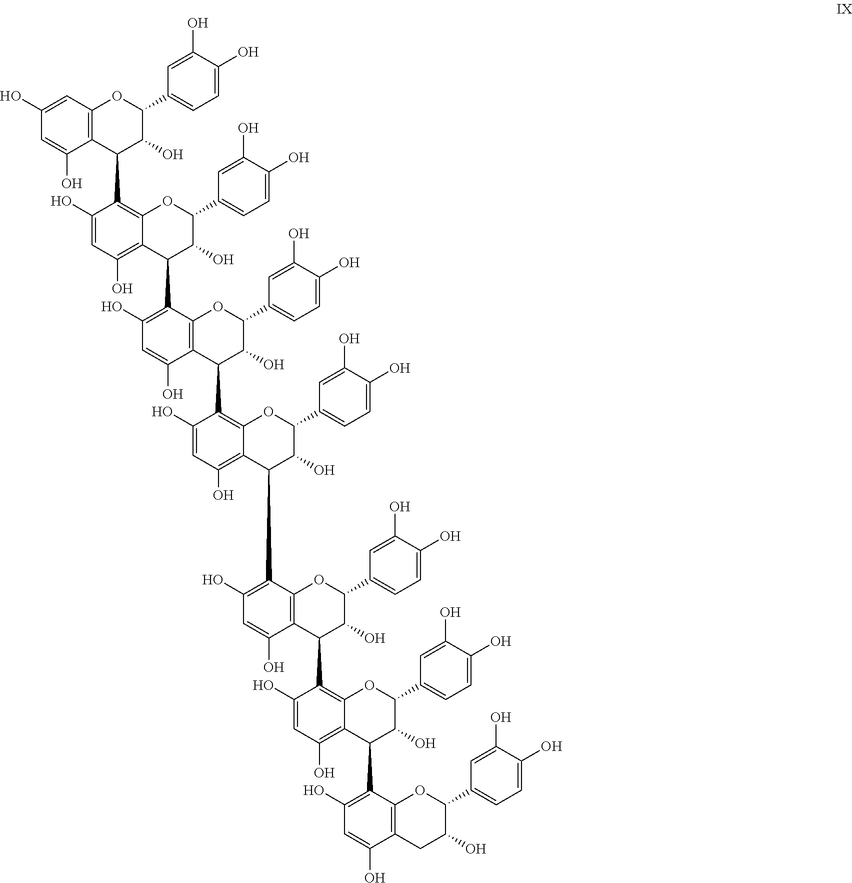

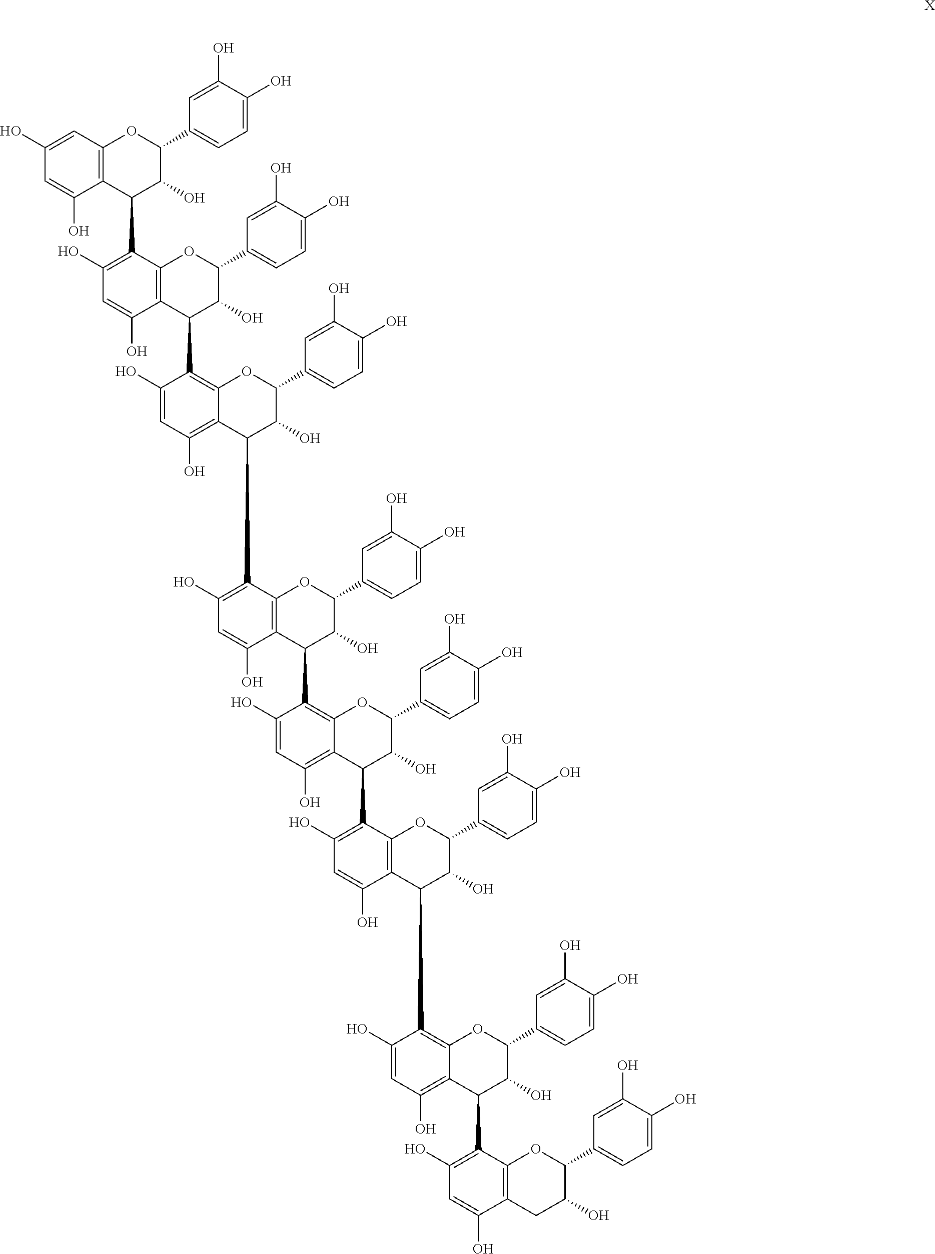

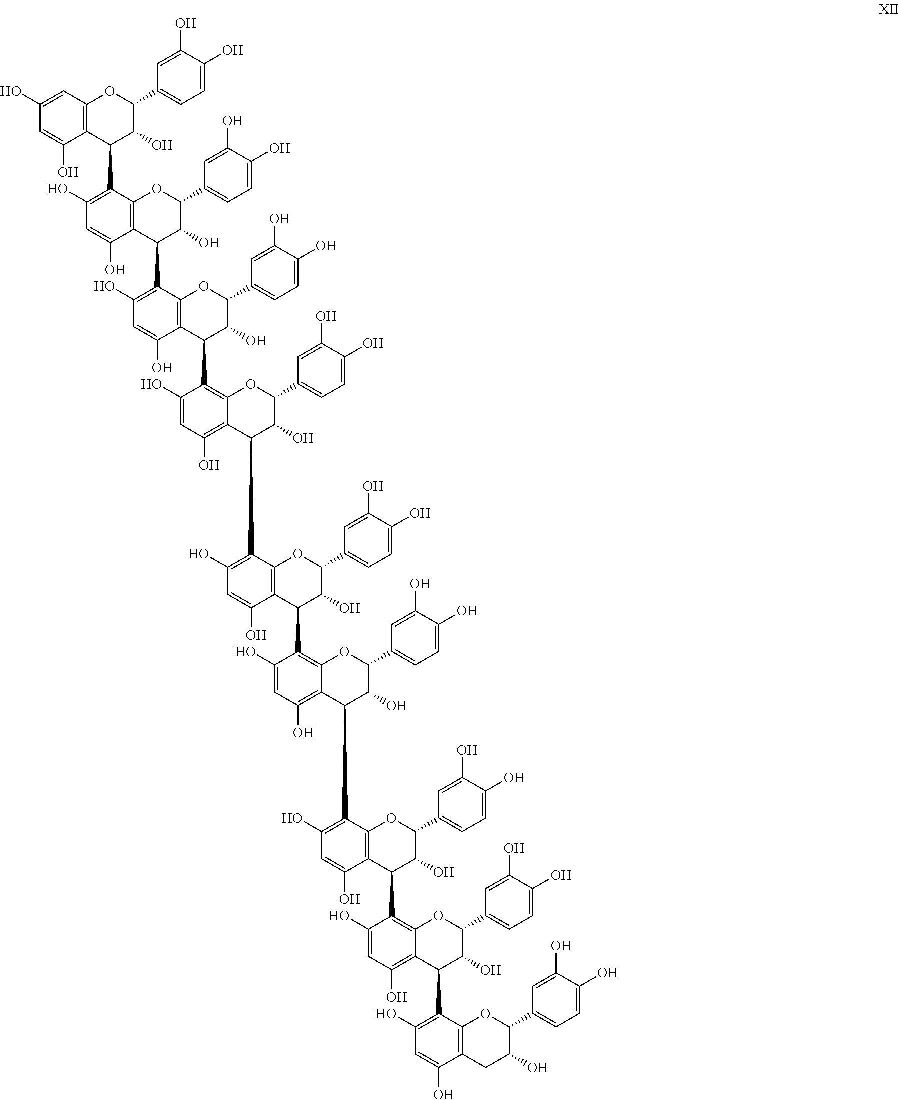

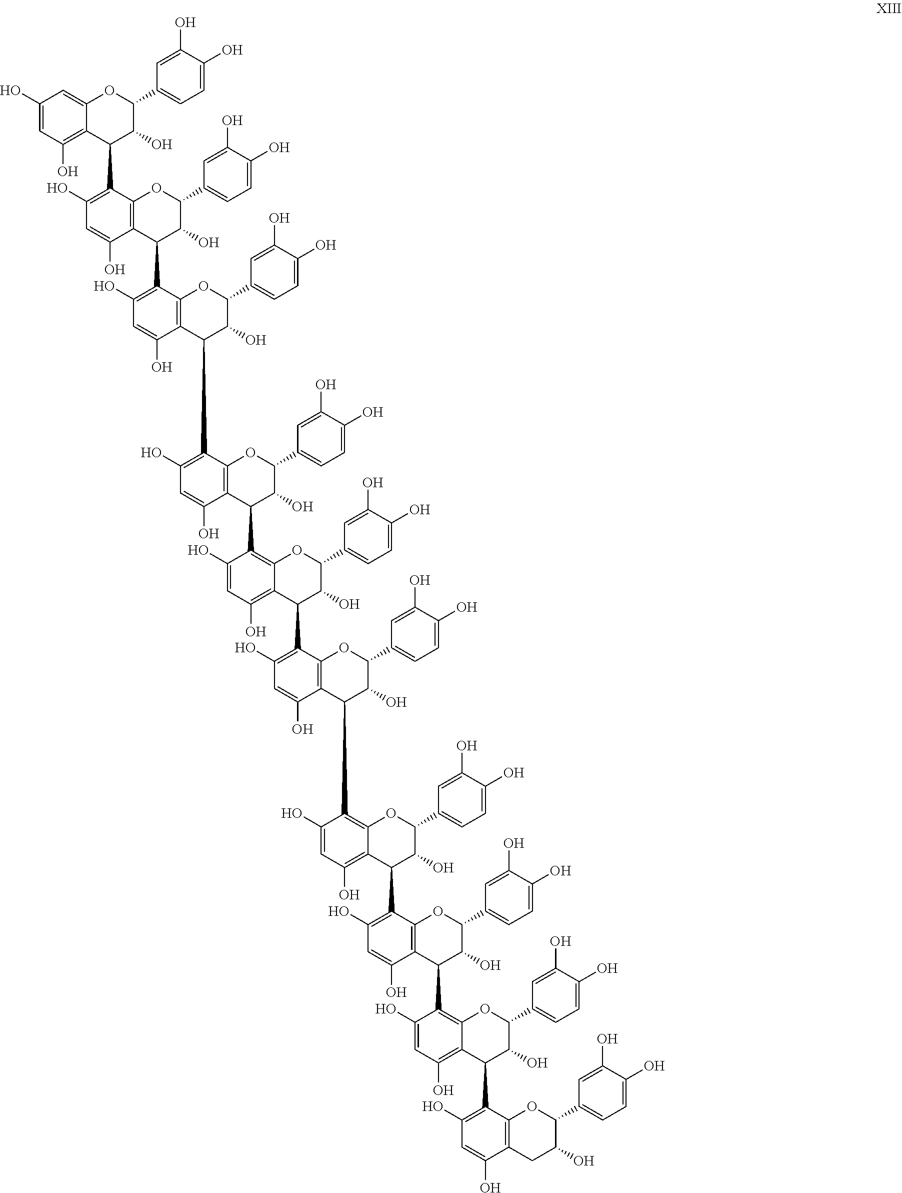

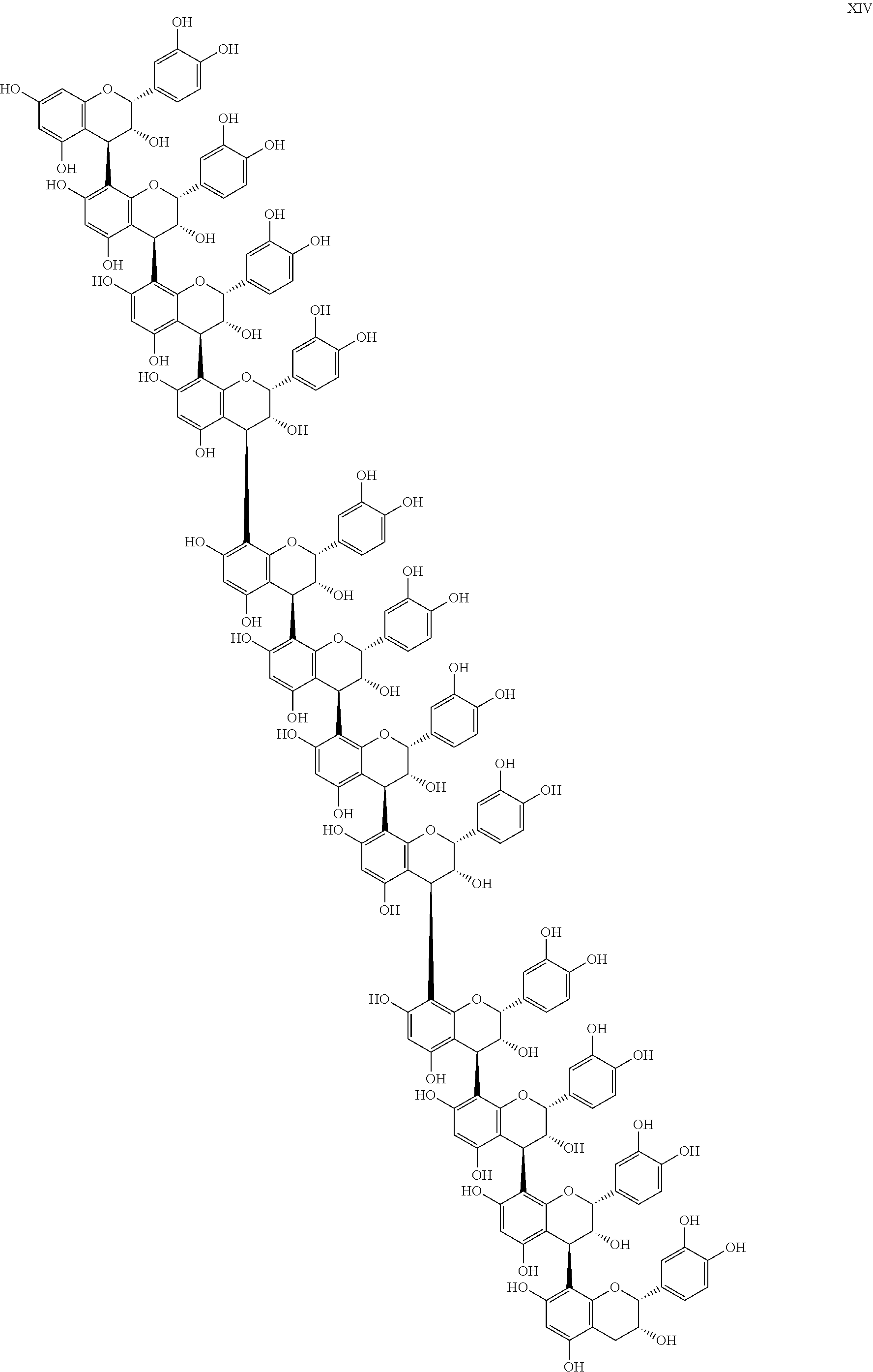

This also includes epigallocatechin anlogues of the compounds IV-XIV supra, exemplified by in a nonlimiting manner XXV-XXI below:

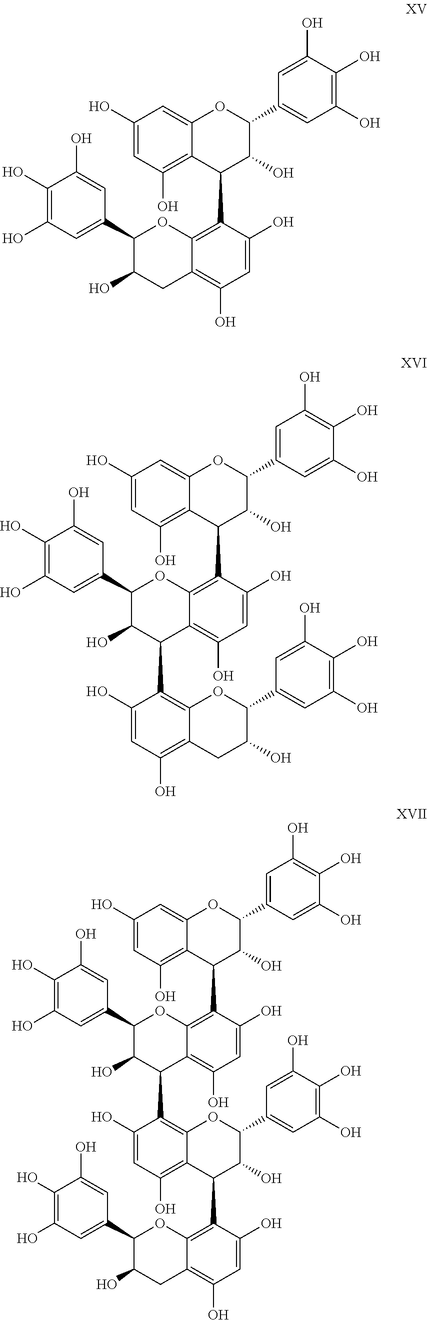

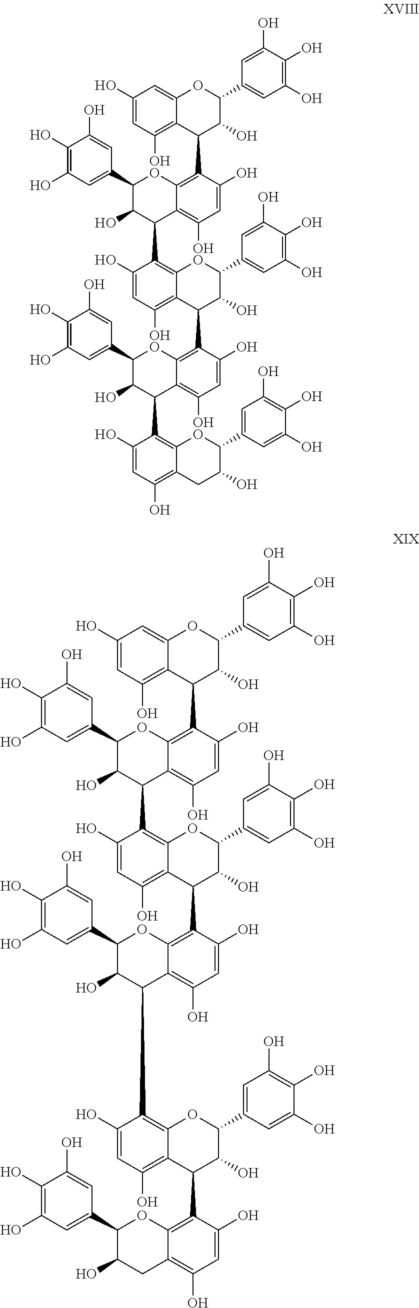

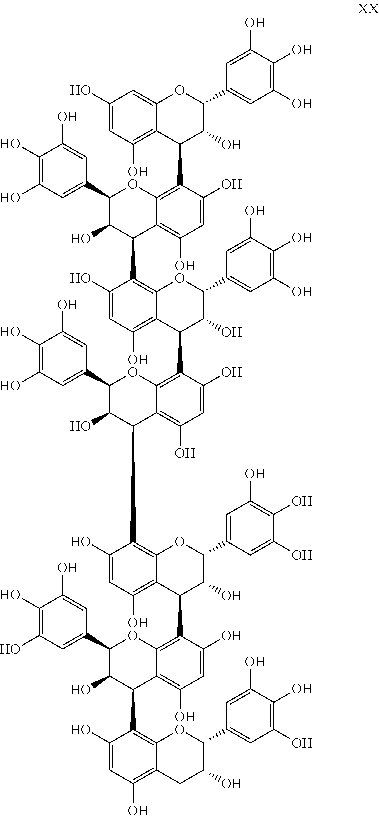

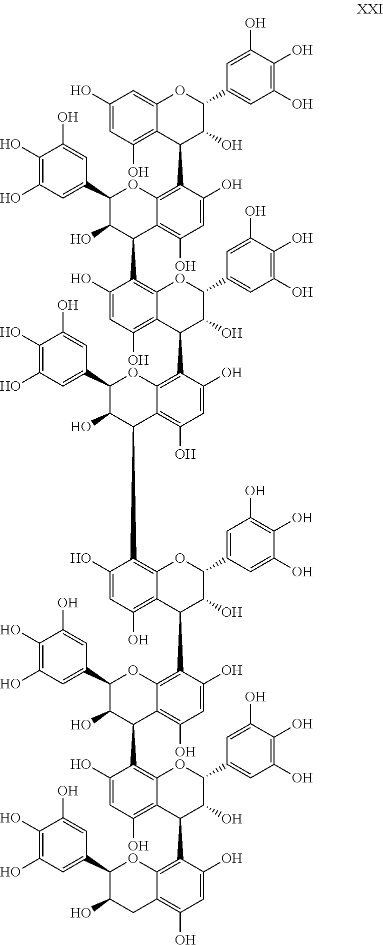

As used herein, the term “alkyl” encompasses linear or branched structures and combination thereof, having the indicated number of carbon atoms. Thus, for example, C₍₁₋₆₎alkyl includes methyl, ethyl, propyl, 2-propyl, s- and t-butyl, butyl, panty, hexyls, 1.1-dimethylethyl, cyclopropyl, cyclobutyl, cyclopentyl and cyclohexyl.

As used herein the compounds of the invention may have one or more asymmetric centers. Compounds with asymmetric centers give rise to enantiomers (optical isomers), diastereomers (configurational isomers) or both, and it is intended that all of the possible enantiomers and diastereomers in mixtures and as pure or partially purified compounds are included within the scope of this invention. The present invention is meant to encompass all such isomeric forms of the epicatechin oligomers supra. Some formulae are shown above without a definite stereochemistry at certain positions. The present invention includes all stereoisomers of the epicatechin oligomers and pharmaceutically acceptable salts thereof.

The independent syntheses of the enantiomerically or diastereomerically enriched compounds, or their chromatographic separations, may be achieved as known in the art by appropriate modification of the methodology disclosed herein. Their absolute stereochemistry may be determined by the x-ray crystallography of crystalline products or crystalline intermediates that are derivatized, if necessary, with a reagent containing an asymmetric center of known absolute configuration. If desired, racemic mixtures of the compounds may be separated so that the individual enantiomers or diastereomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods using chiral stationary phases, which methods are well known in the art. Alternatively, any enantiomer or diastereomer of a compound may be obtained by stereoselective synthesis using optically pure starting materials or reagents of known configuration by methods well known in the art.

The term “pharmaceutically acceptable” means that the carrier, diluent or excipient must be compatible with the other ingredients of the formulation and not deleterious to the recipient thereof.

The terms “administration of or “administering a” compound should be understood to mean providing a compound of the invention to the individual in need of treatment in a form that can be introduced into that individual's body or topically into the individual's dermis in a therapeutically useful form and therapeutically useful amount.

The terms “effective amount” or “therapeutically effective amount” means the amount of the subject compound that will elicit the biological or medical response of a tissue, system, animal or human that is being sought by the researcher, veterinarian, medical doctor or other clinician.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

As used herein, the term “pharmaceutically acceptable salts” encompasses both the metallic (inorganic) salts and organic salts; a list of which is given in Remington's Pharmaceutical Sciences, 17th Edition, pg. 1418 (1985). It is well known to one skilled in the art that an appropriate salt form is chosen based on physical and chemical stability, flowability, hydroscopicity and solubility. As will be understood by those skilled in the art, pharmaceutically acceptable salts include, but are not limited to salts of inorganic acids such as hydrochloride, sulfate, phosphate, diphosphate, hydrobromide, and nitrate or salts of an organic acid such as malate, maleate, fumarate, tartrate, succinate, citrate, acetate, lactate, methanesulfonate, p-toluenesulfonate or pamoate, salicylate and stearate. Similarly pharmaceutically acceptable cations include, but are not limited to sodium, potassium, calcium, aluminum, lithium and ammonium (especially ammonium salts with secondary amines). salts may also be obtained with bases such as ammonium hydroxide or secondary or tertiary amines (such as diethylamine, triethylamine, piperidine, piperazine, morpholine) or with basic amino-acids, or with osamines (such as meglumine) or with amino-alcohols (such as 3-aminobutanol and 2-aminoethanol. Preferred salts of this invention include potassium, sodium, calcium and ammonium salts. Salts in the solid form may exist in more than one crystal structure, and may also be in the form of hydrates

The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefinetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones

The term “anti-viral agent” as used herein means any of a group of chemical substances having the capacity to inhibit the replication of or to destroy viruses used chiefly in the treatment of viral diseases. Anti-viral agents include, but are not limited to, Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Sorivudine, Stavudine, Trifluridine, Valacyclovir, Vidarabine, Zalcitabine, Zidovudine, Acemannan, Acetylleucine, Amantadine, Amidinomycin, Delavirdine, Foscamet, Indinavir, Interferons (e.g., IFN-alpha), Kethoxal, Lysozyme, Methisazone, Moroxydine, Nevirapine, Podophyllotoxin, Ribavirin, Rimantadine, Ritonavir2, Saquinavir, Stailimycin, Statolon, Tromantadine, Zidovudine (AZT) and Xenazoic Acid

Suitable carriers for epicatechin oligomers include water, alcohols, oils and the like, chosen for their ability to dissolve or disperse the active ingredients at concentrations of active ingredients most suitable for use in the therapeutic treatment. Generally, even low concentrations of active ingredients in a carrier will be suitable, even as low as 0.1% by weight. As a practical matter, however, to avoid the need for repeated application, it is desirable that the topically applied composition be formulated to contain at least about 0.25% to about 5% by weight, more preferably from about 1% to about 3% by weight epicatechin oligomers, and accordingly, carriers will be chosen which can solubilize or disperse the active ingredients at such concentrations. Many preferred embodiments contain over 1%, and many over 1.5% by weight epicatechin oligomers

### EXAMPLES

### Example 1

### Production of Epicatechin Oligomers Extract

50 Kg. of Single-source Organic Cocoa Nibs obtained from the Dominican Republic are powdered in a Mill to an average size of 150 mesh. Care is taken not to heat the product during this milling process. A jacketed 200 L reaction vessel is charged with and 70 L of hexane (Baker reagent) are added. The kettle is heated to reflux under argon for a 24-hour period. The vessel is rapidly stirred by a Lightenin-type mechanical stirrer during this period. At the end of this time, hexane is removed by aspiration and the resultant mass is dried in a Buchi 50 L rotary evaporator at 50 C overnight. The resultant hexane fraction is filtered through activated charcoal and recovered by distillation in the above rotary evaporator.

The dry hexane-extracted powder is then placed in a 200 L jacketed reaction vessel and the vessel is charged with 60% acetone (Baker reagent)-40% distilled water. The kettle is heated to reflux under argon for a 24-hour period. The vessel is rapidly stirred by a Lightenin-type mechanical stirrer during this period. At the end of this time, the aqueous acetone is removed by aspiration, and is flash-filtered through chromatographic-grade silica (Baker) and solvent removed in a 50 L Buchi Rotary evaporator to obtain a brown tarry product. This is taken up with absolute ethanol and re-evaporated to yield a semi-crystalline powder. This powder is termed Extract M-1.

1 Kg of the powder M-1 is dissolved in 4 L of 10 mM ammonium acetate containing 20% ethanol, and soluble polyvinylpyrrolidone (500 gm) is added. A flocculent precipitate is obtained which is removed through filtration through diatomaceous earth. The solvent is removed on the rotary evaporator with the aid of 2 L of 2-propanol. Then the dry product is dissolved in 4 L of 50 mM citrate buffer (potassium counterion), pH 3.45, containing 10% (v/v) ethanol. This is poured on a short column packed with 6 Kg of DEAE cellulose (DE-53, Whatman, Inc.) which is equilibrated with the same buffer. With the aid of a peristaltic pump, the column is eluted with a gradient beginning with the starting buffer and ending with 2M ammonium acetate, pH 2.74. Fractions (1 L) are taken. It is determined using an HP LC/MS that samples eluting at 200 mM-400 mM ammonium acetate contain the maximum amount of epicatechin oligomers, in the form of dimmer, trimer, and tetramer. These data are illustrated in FIG. 1. The various supernatants are placed evaporated to dryness at 50 C on a Buchi rotary evaporator. Upon reaching dryness, the temperature is elevated to 80 C and the vacuum is reduced to ca. 30 microns of mercury pressure (oil pump with a liquid nitrogen trap), whereupon the ammonium acetate is removed, although a small amount of residual potassium citrate remains in the extract

### Example 2

### Large Scale Batch Production of Epicatechin Oligomer Extract

20 Kg of the powder M-1 as defined supra is dissolved in 80 L of 10 mM ammonium acetate containing 20% ethanol, and soluble polyvinylpyrrolidone (10 Kg) is added. A flocculent precipitate is obtained which is removed through filtration through a filter press containing diatomaceous earth. The clear supernatant material is placed in a 200 L reaction vessel and sparged with argon. To the supernatant (75 L) is added 750 gm of citric acid (based on anhydrous weight) and using a lightenin stirrer this is put into solution and 10 liters of deionized water is added. The pH is adjusted to 3.45. with 12 M KOH. To this vessel is then added 70 Kg. of DEAE Cellulose (DE-52, Whatman) which has been preequilibrated with 50 mM citrate buffer (potassium counterion), pH 3.45, containing 10% (v/v) ethanol. This mixture is gently stirred for one hour under argon at room temperature. Then, the stirrer is turned off and the resin is allowed to settle. After two hours, the resin is settled to the bottom of the reaction vessel and the supernatant is removed be decantation leaving the wet resin at the bottom. The supernatant is discarded. To the reaction vessel containing the wet resin is added 100 L of 50 mM citrate buffer (potassium counterion), pH 3.45. This is stirred for one hour, then the stirrer is turned off, and the buffer is decanted and discarded. To the washed wet resin is then added 70 L of 200 mM ammonium acetate, pH 2.47, containing 10% (v/v) ethanol. This is stirred in the above manner for two hours, stirring is removed, and the supernatant is removed and retained. This is called the S-1 supernatant. Then, 70 L of 400 mM ammonium acetate, pH 2.47, containing 12% (v/v) ethanol, is added, and is stirred for two hours, stirring is removed, and the material allowed to settle. This is referred to as S-2. Supernatant S-1 and S-2 are combined and the pH is adjusted to 7.4 with 12 M KOH. The combined supernatants are placed evaporated to dryness at 50 C on a 50 L Buchi rotary evaporator. Upon reaching dryness, the temperature is elevated to 80 C and the vacuum is reduced to ca. 30 microns of mercury pressure (oil pump with a liquid nitrogen trap), whereupon the ammonium acetate is removed, although a small amount of residual potassium citrate remains in the extract.

### Example 3

### Demonstration of Antiviral Activity of the Epicatechin

### A. HPV Assay

The Theobroma cacao epicatechin oligomer compounds of the invention are also useful as tools to probe the HPV life cycle.

Human keratinocytes, including those maintaining HPV episomes, are cultured on mitomycin C-treated J2 3T3 cells in media containing three parts Dulbecco's modified Eagle medium (DMEM) and one part F12 media. This media mixture is supplemented with 0.4 ug/mL hydrocortisone, 10 ng/mL cholera toxin, 5 ug/mL insulin, 24 ug/mL adenine, 5 ug/mL transferrin, 5 ug/mL 3,3[prime], 5-triiodo-thyronine (T(3)), 10 ng/mL epidermal growth factor (EGF), 1% penicillin-streptomycin, and 5% fetal bovine serum (FBS). All cells are passaged at 70% confluency.

Theobroma cacao epicatechin oligomers are dissolved at 10 mM in 100% DMSO and diluted with H2O to 1 mM. Theobroma cacao epicatechin oligomers are added to cells in the above media at final concentrations of 0.1-10 uM, As controls, cells are incubated with normal E media and E media containing 0.1% DMSO vehicle. The HPV DNA levels are then quantified according to previously published procedures. After incubation, cells are harvested from the plates by either trypsinization or direct lysis with proteinase K digestion buffer (100 mM NaCl, 10 mM Tris pH 8, 25 mM EDTA, 0.5% SDS, 0.1 mg/mL proteinase K). Trypsinized cells are counted on a hemocytometer and pelleted by centrifugation. Episomal HPV is isolated and cell pellets are lysed in 0.6% SDS. NaCl is next added to a final concentration of 1 M. Following an overnight incubation at 4 C., precipitates containing the chromosomal DNA are sedimented at 100,000×g and episomal DNA precipitated by the addition of isopropanol. Cells lysed directly in proteinase K buffer are transferred to microfuge tubes and incubated at 50 C. for 2 h. Lysates are then extracted with phenol/chloroform/isoamyl alcohol and spun through a phase lock gel. Total DNA is then precipitated with 0.3 M NaOAc and 3 v/v ethanol and resuspended in Tris-EDTA (TE) buffer, pK 7.40.

Viral DNA levels are next quantified using RT-PCR on an ABI PRISM 7700 Sequence Detector. For HPV 18, PCR primer-probe sets were designed within the L1 gene: sense 5′-TTTGGTTCAGGCTGGATTGC (SEQ ID NO: 1), antisense 5′-GCAGATGGAGCAGAACGTTTG (SEQ ID NO: 2), probe 5′-TCGCAAGCCCACCATAGGCCC (SEQ ID NO: 3). HPV31 primers-probe sets for PCR were also designed within the L1 gene: sense 5′-CTGCTATTTTGGAAGATTGGAAT (SEQ ID NO: 4), antisense 5′-GGCCTGTGAGGTGACAAACC (SEQ ID NO: 5), probe 5′-TTGGATTGACCACACCTCCCTCAGGTT (SEQ ID NO: 6). All primers and probes are synthesized and HPLC purifiedcommercially. The HPV probes are labeled with the 5′-reporter dye FAM (6-carboxy-fluorescein) and the 3′-quencher dye TAMRA (6-carboxytetramethyl-rhodamine). A standard curve is generated using genomic HPV31 DNA using the following formula: (1.82×10(15))(ug/uL stock DNA)/(length in base pairs)×(2)=copies/uL stock DNA. PCR reactions contain final concentrations of 1× Universal Master Mix (PE Applied Biosystems), 200 nM of each primer, and 300 nM probe (PE Applied Biosystems) in a reaction volume of 25 uL. Each DNA sample is analyzed in triplicate reactions for episomal HPV. Copies/reaction are determined from the standard curve, and copies/cell determined according to the following formula: (copies/reaction)×(DNA dilution)/(total # cells)=copies/cell.

### B. In Vitro Toxicity Assessment

Theobroma cacao epicatechin oligomers that significantly reduce HPV18 DNA levels can be further tested in a series of follow up studies. Southern blotting is used to confirm the effects of Theobroma cacao epicatechin oligomers on HPV 18 DNA levels that were determined using real-time PCR technology. Briefly, 5 ug of total cell DNA from both Theobroma cacao epicatechin oligomer-treated and control cells is digested with BamHI and run on a 0.7% agarose gel. After transfer to Nytran membranes, the DNA is probed with gel purified full-length HPV18 that has been liberated from pUC19 with BamHI, and randomly primed in the presence of DIG-UTP (Roche). Following incubation with anti-DIG AP (alkaline phosphatase), HPV DNA is detected with ECF substrate and phosphor-imaging.

50% and 90% effective concentration values (EC(50)& EC(90)) are determined for each Theobroma cacao epicatechin oligomer over a dose range of 10 nM to 500 uM. The final levels of HPV DNA per cell are determined for each Theobroma cacao epicatechin oligomer concentration, and data is expressed as % inhibition relative to vehicle-treated controls.

The toxicity of each Theobroma cacao epicatechin oligomer found active against HPV18 is monitored in normal human keratinocytes using a standard MTT cell viability assay. Theobroma cacao epicatechin oligomers are initially supplied to normal keratinocytes in growth media at concentrations of 10 nM, 100 nM, 1 uM, 10 uM, 100 uM, 500 uM, 1 mM and 10 mM. Each set of samples is supplied in triplicate, in clear 96-well plates. A tetrazolium dye (3-[4,5-dimethylthiazol-2-yl]-2,5-diphenyl tetrazolium bromide or MTT) is added to the cell cultures 48 hours after addition of Theobroma cacao epicatechin oligomers. After 4 hours cells are rinsed once with PBS, and isopropanol containing 0.04N HCl is added to lyse cells and solubilize the MTT formazan. Plates are read on a plate reader at a test wavelength of 570 nm and a reference wavelength of 630 nm. Data are expressed as % inhibition of vehicle-treated controls and, as for analysis of effects on HPV DNA levels, IC(50)s are calculated using nonlinear regression analysis.

The therapeutic index for the Theobroma cacao epicatechin oligomesr is then determined as the ratio of the EC(50) to the IC(50)(SI=EC(50)/IC(50)).

Finally, the effects of multiple dosing with Theobroma cacao epicatechin oligomers are followed in vitro. The purpose of these studies is to gauge the extent to which Theobroma cacao epicatechin oligomers can clear cells of episomal DNA in the absence of an immune system. While it is recognized that, in general, an intact immune system is important for optimal antiviral effects, these studies are important to help prioritize and select compounds designed to clear viral DNA in animal studies. Since typical clinical antiviral regimens last from 1 to 2 weeks or longer, we can dose HPV18-positive keratinocytes for 6, 9, and 12 days by providing fresh Theobroma cacao epicatechin oligomer with each change of medium. The HPV-18 keratinocytes are passaged during the course of these experiments as needed. Dosage can be at levels >EC(90) value as long as those concentrations previously showed no significant toxicity. Treated cells are then collected for PCR analysis and also re-plated in fresh media. The re-plated cells are allowed to recover for an additional 7 days at which time they are harvested and viral DNA content analyzed by PCR. The viral DNA content of the recovered cells is then compared with that of the cells at the end of the treatment regiment.

The data for HPV show a remarkable and potent reduction in viral DNA levels.

### Example 4

Treatment of patients with epicatechin mixture following laser surgery for genital warts.

Patients (N=24) received laser surgery in the labial, vaginal, or cervical area for removal of genital warts. Half (N=12) of these patients were treated with a preparation containing 1% (w/v) of epicatechin oligomers in a polyethylene glycol base containing 1% lidocaine. The other half (N=12) were treated with the same preparation lacking the epicatechin oligomers. The results are illustrated below:

| GROUP | Mean Days to Healing (sem) | Severity Score at Day 2(1-10) |
|---|---|---|
| treated | 6(2) | 2.5 |
| control | 10(3) | 6 |

What is claimed is:

1. A method for acceleration of healing of genital warts by applying highly purified epoicatechin oligomers from Theobroma Cacao, wherein such healing is accelerated by a combination of the antiviral and antioxidant properties of the highly purified epicatechin oligomers.

2. The method according to claim 1, further comprising extirpation of said genital wart prior to said applying, carried out by use of low temperatures, in the form of cryosurgery.

3. The method according to claim 1, further comprising extirpation of said genital wart prior to said applying, wherein extirpation of said genital wart is carried out by means of an elevated temperature cautery, either of a monopolar or bipolar nature, or by means of a heated wire, which may or may not be attached to an endoscopic device.

4. The method according to claim 1, further comprising extirpation of said genital wart prior to said applying, wherein extirpation of said genital wart is carried out by means of a laser.

5. The method according to claim 1, whereby such defined epicatechin oligomers are in the form of a kit for one of intravaginal, intrarectal, and intracervical use.

6. The method according to claim 1, wherein the mixture of defined antiviral epicatechin oligomers is contained in a base which is a hydrophylic gel, adjusted to appropriate pH for intravaginal application

7. The method of claim 1, wherein such healing is accelerated by antioxidant properties of the highly purified epicatechin oligomers.

8. The method of claim 1, wherein such healing is accelerated by antiviral properties of the highly purified epicatechin oligomers

9. A method of inhibiting virus replication in a mammal comprising administering to said mammal an anti-viral amount of a compound of formula an antiviral epicatechin oligomer.

10. The method according to claim 9, wherein said virus is selected from the group consisting of HIV, HPV, HBV, HCV, HSV-1, HSV-2, Parainfluenza, Influenza A Influenza B, Adenovirus, RV smallpox, varicella virus, coronavirus, and RVS.

11. The method of claim 1, wherein said epicatechin oligomers is about 0.1% to about 5.0% by weight of said composition.

12. The method of claim 1, wherein said epicatechin oligomers is about 0.25% to about 3.0% by weight of said composition.

13. The method of claim 1, wherein said epicatechin oligomers is about 0.5% to about 20.0% by weight of said composition.

14. The method of claim 1, wherein an amount of said catechins and/or catechins salts or esters or is about 0.1% to about 5.0% by weight.

15. The method of claim 1, wherein an amount of said catechins and/or catechins salts or esters or C4 to C18 esters is about 0.5%-20.5% by weight of said composition.

16. The method of claim 1, wherein said composition further comprises one or more additional ingredients selected from the group consisting of: tocotrienols and vitamin E compositions enriched with tocotrienols.

17. The method of claim 1, wherein said composition further comprises one or more additional ingredients selected from the group consisting of ergothioneine, lipoic acid, ovothiol, cysteine, penicillamine, N-acetylcysteine, cysteine C₁-C₃₀ alkyl ester, ebselen, sodium selenite, AD-4 thiol antioxidant, homocysteic acid, buthionine sulfoximine, selenocysteine, selenomethionine, bucillamine, N-acetylcysteine amide, 1,2-dithiol-3-thione, pyrrolidine dithiocarbamate, alkyl-2-thioacetate ester, alkyl 3-thiopropionate alkyl ester, alkyl-2-thiolpropionate alkyl ester, 3-(p-methoxyphenyl)-1,2-dithiol-3-thione; L-2-oxathiazalidine-4-carboxylate, alkyl-2-thiobutanoic ester, alkyl-4-thiobutanoic ester

18. The method of claim 16, wherein an amount of said additional ingredient is about 0.1% to about 5.0% by weight of said composition.

19. The method of claim 1, wherein said composition further comprises one or more additional antibiotic ingredients selected from the group consisting of Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefinetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, muprocin, and fluoroquinolones

20. The method of claim 1, wherein said composition further comprises one or more additional antivirals selected from the group consisting of: Abacavir, Acyclovir, Adefovir, Amantadine, Amprenavir, Ampligen, Arbidol, Atazanavir, Atripla, Berberine, Boceprevir, Chelythrine, Cidofovir, Combivir, Darunavir, Delavirdine, Didanosine, N,N-Dioctadecyl-N′,N′-Bis(2-Hydroxyethyl) propanediamine, Docosanol, Edoxudine, Elvucidabine, Efavirenz, Emtricitabine, Enfuvirtide, Entecavir, Famciclovir, Fomivirsen, Fosamprenavir, Foscarnet, Fosfonet, Ganciclovir, Helioxanthin, Ibacitabine, Immunovir, Idoxuridine, Imiquimod, Indinavir, Inosine, Lamivudine, Lopinavir, Loviride Maraviroc, 1′-methyl spiro (adamantane-2,3′-pyrrolidine) maleate, Moroxydine, Nelfinavir, Nevirapine, Nexavir, Oseltamivir, Peginterferon alfa-2a, Penciclovir, Peramivir, Pleconaril, Podophyllotoxin, Raltegravir, Ribavirin, Rimantadine, Ritonavir, Saquinavir, Stavudine, Tenofovir, Tipranavir, Trifluridine, Trizivir, Tromantadine, Truvad, Valaciclovir, Valganciclovir, Vicriviroc, Vidarabine, Viramidine, Zalcitabine, Zanamivir, Zidovudine, (+)-(1S,4R)-9-[2,3-Dideoxy-2,3-didehydro-3-fluoro-6-hydroxymethylcyclopent-2-enyl]guanine, (+)-(1S,4R)-9-[2,3-Dideoxy-2,3-didehydro-3-fluoro-6-hydroxymethylcyclopent2-enyl]thymine, (−)-(1S,4R)-9-[2,3-Dideoxy-3,3-difluoro-6-(0-tert-butyl-diphenylsilyloxymethyl)-cyclopentanyl]adenine, N-9, octoxynol-9, benzalkonium chloride, chlorhexidine, (5-[(4-bromophenyl)methyl]-2-phenyl-5H-imidazo[4,5-c]pyridine, 5-Ethyl2′-deoxyuridine 5-vinyl-2′-deoxyuridine, 5-propyl-2′-deoxyuridine, 5-allyl-2′-deoxyuridine, 3′-octanoyl-2,2′-anhydro-1-beta-D-arabinofuranosylcytosine, 3′-decanoyl-2,2′-anhydro-1-beta-D-arabinofuranosylcytosine, hexadecyloxypropyl ester of 9-(5-phosphono-pent-2-en-1-yl)-adenine, N-methanocarbathymidine, ((2s)-2-[(2,4-dichloro-benzoyl)-(3-trifluoromethyl-benzyl)-amino]-3-phenyl-propronic acid, (3-benzylidenechroman-4-one, 3-benzyl-4-chromone, 3-benzylchroman-4-one, 3-methyleneoxindole, mersalyl, 2-Amino-5-bromo-6-methyl-4-pyrimidinol, or 5-Methoxycarbonyl-6-methyl-4-(5-nitrofuryl)-2-oxo-1,2,3,4-tetrahydropyrimidine


SEQUENCE LISTING (6 sequences)

SEQ ID NO: 1 - DNA, 20 bp, Artificial Sequence
  Description of Artificial Sequence Synthetic primer
tttggttcaggctggattgc

SEQ ID NO: 2 - DNA, 21 bp, Artificial Sequence
  Description of Artificial Sequence Synthetic primer
gcagatggagcagaacgtttg

SEQ ID NO: 3 - DNA, 21 bp, Artificial Sequence
  Description of Artificial Sequence Synthetic probe
tcgcaagcccaccataggccc

SEQ ID NO: 4 - DNA, 23 bp, Artificial Sequence
  Description of Artificial Sequence Synthetic primer
ctgctattttggaagattggaat

SEQ ID NO: 5 - DNA, 20 bp, Artificial Sequence
  Description of Artificial Sequence Synthetic primer
ggcctgtgaggtgacaaacc

SEQ ID NO: 6 - DNA, 27 bp, Artificial Sequence
  Description of Artificial Sequence Synthetic probe
ttggattgaccacacctccctcaggtt